(19) United States
(12) Patent Application Publication
(10) Pub. No.: US 2002/0111311 A1
(43) Pub. Date: Aug. 15, 2002

(54) Daptomycin and related analogs in crystalline form

(76) Inventors: Chandrika Govardhan, Lexington, MA (US); Nazer Khalaf, Worcester, MA (US)

Correspondence Address: MINTZ, LEVIN, COHN, FERRIS,, GLOVSKY and POPEO, P.C., One Financial Center, Boston, MA, 02111, US

(21) Appl. No.: 10/024,405

(22) Filed: Dec. 18, 2001

Related U.S. Application Data
(60) Provisional application No. 60/274,741, filed on Mar. 9, 2001.
(60) Provisional application No. 60/256,268, filed on Dec. 18, 2000.

(51) Int. Cl. A61K 38/17; C07K 7/64

(52) U.S. Cl. 514/12; 530/359; 530/317; 514/9

(57) ABSTRACT

The invention provides the cyclic lipopeptide daptomycin and A-21978C analogs in crystalline form, methods of preparing the crystalline forms, compositions comprising the crystalline forms and methods of using these forms.

### CROSS-REFERENCE TO RELATED APPLICATIONS

The present application claims the benefit of U.S. Provisional Application No. 60/274,741, filed Mar. 9, 2001 and U.S. Provisional Application No. 60/256,268, filed Dec. 18, 2000. The contents of those applications are hereby incorporated herein in their entirety.

### TECHNICAL FIELD OF THE INVENTION

The present invention relates to methods of crystallizing low molecular weight polypeptides, in particular the low molecular weight cyclic lipopeptide antibiotic daptomycin and related A-21978C analogs.

### BACKGROUND OF THE INVENTION

As disclosed in U.S. Pat. No. 4,482,487, the A-21978C antibiotics are a complex of closely related, lipopeptides with potent gram-positive antibacterial activity. The complex is characterized by a mostly cyclic structure of 13 amino acids bound to a fatty acid moiety in the side chain. The individual factors of the complex, C₀, C₁, C₂ , C₃, C₄, and C₅, are distinguished by the make-up of the fatty acid side chain moiety. Factor A-21978C₀ is itself a complex of two compounds, a major component comprising a branched C-10 alkanoyl side chain, and a minor component comprising an n-decanoyl side chain, which is known as daptomycin (U.S. Pat. No. 5,912,226) (see, FIG. 1). Daptomycin, in an injectable formulation known as CIDECIN® (Cubist Pharmaceuticals, Inc.), is currently in clinical trials for treatment of serious and life-threatening gram-positive pathogen-related diseases including complicated skin and soft tissue infections, community-acquired pneumonia, and complicated urinary tract infections and is to said to be entering clinical research for treatment of enteroccocal infections, endocarditis and bacteremia.

Crystal forms of therapeutic peptides are highly desirable for their stability during storage, improved purity, bulk crystallization product manufacture, and in the process of formulating pharmaceutical compositions. This stability is known to aid in the effectiveness of the peptides when ultimately administered. In addition, production of crystalline forms of peptides, whether dried or in solution, allows for higher purity therapeutic compositions, as well as provide for higher concentration solutions of such compositions when compared to amorphous precipitate forms. No crystal form of A-21978C cyclic peptide antibiotics has heretofore been described. Thus, a method of preparing the A-21978C antibiotics, including daptomycin, in crystal form would be useful.

### SUMMARY OF THE INVENTION

The invention provides methods for producing crystalline forms of lipopeptides particularly daptomycin and daptomycin-related lipopeptides. In another embodiment, the invention provides a method for purifying lipopeptides by crystallization and/or precipitation. In a further embodiment, the invention provides for pharmaceutical compositions comprising the crystalline forms that can be used in alternate modes of delivery and control of dosage.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are hereby incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 shows the structure of daptomycin.

FIG. 2 shows a photomicrograph of urchin-like crystals of daptomycin.

FIG. 3 shows a photomicrograph of needle-like crystals of daptomycin.

FIG. 4 shows a photomicrograph of rod-like crystals of daptomycin.

FIG. 5 shows photomicrographs of daptomycin samples at 100X magnification. Photomicrographs of amorphous daptomycin are shown using plane transmitted light (A) and using crossed polarized light (B). Photomicrographs of daptomycin crystals are shown using plane transmitted light (C and E) and using crossed polarized light (D and F). The daptomycin crystals were produced by the protocol disclosed in Example 6.

FIG. 6 shows a photomicrograph of urchin-like crystal or crystal-like particle of daptomycin produced by the method described in Example 8.

FIG. 7 shows birefringence of a crystal-like particle of daptomycin produced by the method described in Example 8 that was exposed to polarized light.

### DETAILED DESCRIPTION OF THE INVENTION

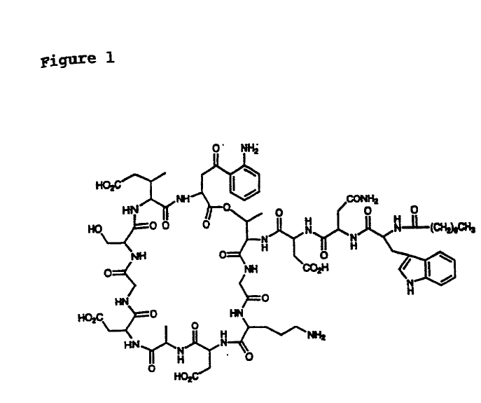

This invention provides a low molecular weight cyclic peptide in crystal form. In a preferred embodiment, the low molecular weight cyclic peptide is chosen from the group consisting of daptomycin and related A-21987C analogs. As used herein, “daptomycin” refers to the compound depicted in FIG. 1, which is the n-decanoyl derivative of the factor A-21978C₀-type antibiotic that contains an α-aspartyl group. Daptomycin is also known as LY 146032. Methods of producing daptomycin are known to those in the art, including those methods described in U.S. Pat. Nos. Re. 31,396, 4,537,717, 4,800,157, and 4,874,843.

As used herein, “A-21978C analog” refers to a compound having the core peptide structure of the cyclic lipopeptides of the A-21978C complex, which includes factors Co, C₁, C₂, C₃, C₄, and C₅, and modified forms thereof, including salts thereof, and all possible stereoisomers thereof. Several patents and published applications describe modified forms of the cyclic lipopeptide A-21978C complex, such as U.S. Pat. Nos. 5,912,226, 4,885,243, 4,537,717, 4,524,135, Re. 32,311, Re. 332,310, and published PCT applications WO 01/44271, WO 01/44272, and WO 01/44274, each published Jun. 21, 2001. These disclosures describe modifications at various positions within the individual peptides of the cyclic lipopeptide structure and modifications of the fatty acid side chain on the complex. All such modifications, as exemplified by the compounds described in these disclosures, are within the scope of “A-21978C analog” as that term is used herein.

As used herein, the term “crystalline form” of the low molecular weight cyclic peptides daptomycin and A-21978C analogs refers to a homogenous solid state of the matter which displays characteristic features of crystals including lattice structure, characteristic shapes and optical properties such as refractive index and birefringence. Crystalline forms of compounds as used herein are those, for example, occurring as dried crystals or existing as crystals in solution. Crystalline forms of a compound are distinguished from amorphous solid forms of the compound which do not have the molecular lattice structure characteristic of the crystalline form and do not display refractive index or birefringence or other optical or spectroscopic properties of crystals. The crystals formed according to the invention may be any shape, for example needle-like, rod-like, needle cluster or urchin-like, flake-like, or plate-like. In a preferred embodiment, the crystals are urchin-like, rod-like or needle-like. The invention is intended to cover crystals in dried form or crystals formed in solution whether or not the crystals in solution retain their original crystalline form when subjected to further processes, such as drying or separation techniques.

Methods of Producing Crystals

This invention also provides a method for producing crystalline forms of a cyclic lipopeptide chosen from the group consisting of daptomycin and A-21978C analogs which comprises contacting the lipopeptide with a crystallization solution comprising at least one polyhydric alcohol and at least one cation. The invention thus also provides a crystallization solution for use in crystallizing daptomycin and A-21978C analogs which comprises daptomycin or other A-21978C analog, at least one low molecular weight or polyhydric alcohol and at least one cation. In a preferred embodiment, the crystallization solution contains at least one cation and at least one low molecular weight alcohol. The low molecular weight alcohol is typically present in an amount from about 2% to about 90%, and preferably in an amount from about 60% to about 88%. The polyhydric alcohol is typically present in an amount from about 2% to about 40%, and preferably in an amount from about 4% to about 15% Examples of low molecular weight or polyhydric alcohols useful in the crystallization solution include, without limitation, methanol, isopropanol, tert-butanol, n-propanol, a diol or glycol such as ethylene glycol, propylene glycol, glycerol, 1,2-propane diol, 2-methyl-2,4-pentanediol, 1,6-hexanediol, and 1,4-butanediol. The cation can be monovalent, divalent, or trivalent cation. In a preferred embodiment, the cation is a divalent cation. Examples of preferred divalent cations include manganese, magnesium, and calcium. In a particularly preferred embodiment the divalent cation is calcium. The cation is generally provided in solution by addition of a salt to the solution. Examples of salts include, without limitation, formates, phosphates, acetates, hydrates, chlorides, chlorates, sulfates, citrates, and combinations thereof. In addition to being a vehicle for adding the cation, certain salts, such as phosphates, acetates, citrates, are also useful for providing a pH buffering effect on the solution. Salts can be added to the crystallization solution at a concentration of from about 0.001 to about 0.5 M In a preferred embodiment, the salt is added to a concentration of from about 0.005 to about 0.2 M.

The crystallization solutions can also contain additional components that may be useful in the forming crystals including organic precipitants, additional pH buffers, additional low molecular weight alcohols, and detergents. Examples of organic precipitants include polyalkylene oxides or polyalkylene glycols such as polyethylene glycol (PEG), including in particular polyethylene glycol-monomethyl ether (PEG-MME), varying in molecular weight between 300 and 10,000 and 2-methyl-2,4-pentanediol. Organic precipitants can be added to the crystallization solution in an amount from about 2% to about 40%, preferably from about 4% to about 15%. Examples of additional pH buffers include Tris, 4-(2-hydroxyethyl)piperazine-1-ethanesulfonic acid (HEPES), 2-morpholinoethanesulfonic acid (MES), sodium borate, sodium cacodylate, imidazole and tri-sodium citrate dihydrate. Examples of additional alcohols include, without limitation, low molecular weight alcohols such as methanol, propanols, and butanols. Buffering agents can be added to a concentration of from about 0.001M to about 0.3M, preferably from about 0.01M to about 0.05M. Examples of detergents include methyltrioctyl ammonium chloride and TritonDF-12, which can be added to the crystallization solution in an amount from 0 to about 2%, preferably from about 0.01% to about 0.05%.

Several general methods of crystallization of compounds are known including, vapor diffusion, free interface diffusion, batch and micro-batch crystallization, and dialysis. In a preferred embodiment the crystals of the invention are formed using vapor diffusion crystallization techniques. In an equally preferred embodiment, the crystals of the invention are formed using batch crystallization techniques. Vapor diffusion, which is also referred to as hanging drop, sandwich drop or sitting drop, makes use of evaporation and diffusion of water between solutions of different concentration to form a supersaturated solution of the sample, leading to crystal formation. For example, in hanging drop vapor diffusion, a drop containing the sample in the crystallization solution is suspended in a sealed container over a reservoir of the crystallization solution at a higher concentration but without sample. Batch and micro-batch crystallization refer to the combination of a concentrated sample and a concentrated crystallization solution to produce a final concentration which is supersaturated in the solute (sample) and leads to crystallization of the sample. Micro-batch crystallization and hanging drop vapor diffusion are useful methods for screening for correct crystallization conditions and crystallization solution component concentrations.

The crystallization methods of the invention are typically carried out in a crystallization solution having a pH of between about 1.5 and 2.0 or between about 3.0 to about 8.5. In a preferred embodiment, the pH of the crystallization solution is between about 5.0 to about 8.5. The crystallization methods of the invention are also typically carried out at a temperature of between about 0° C. and about 30° C. Temperatures may be varied depending on the method of crystallization used and can be varied during the crystallization period. For example, if using the vapor diffusion method of crystallization, the temperature of the crystallization solution may be maintained between about 4° C. and about 20° C. For batch techniques, crystallization may be initiated at room temperature and then proceed for several hours to several days at room temperature (for example from about 20° C. to about 28° C.). Alternatively after initiation, the solution can stored at reduced temperatures, such as below 10° C., such as at 4° C.

Crystallization of the lipopeptides according to the methods of the invention provide a means for obtaining more purified forms of the lipopeptides, for example over amorphous forms of the lipopeptides, or for decreasing or eliminating one or more contaminants from preparations of the peptides. For example, in the preparation of amorphous daptomycin, the peptide is typically obtained in a purity no higher than 97% purity, and in some instances, no higher than 95% purity. In contrast, according to the methods of the invention, crystals can be produced which allows for increasing the purity to above 95% pure and, in a preferred embodiment, above 97% pure. Thus, the invention provides a method of increasing the purity of a sample of lipopeptides such as daptomycin and A-21978C analogs.

Crystals formed according to the methods of the invention can be isolated by methods known to those in the art, such as centrifugation or filtering, and dried or may be utilized in solution when the crystallization solution contains reagents that are pharmaceutically acceptable components. Drying can be performed by known methods, for example, vacuum drying, spray drying, tray drying or lyophilization. In a preferred embodiment, the dried crystals prepared according to the methods of the invention are more exhibit a higher stability than an amorphous form of the lipopeptide as to heat, light, pH, enzymatic degradation or humidity. Stability of the crystalline lipopeptide of the invention can be measured by antibiotic activity or degradation of the lipopeptide.

In another embodiment, the crystalline lipopeptide is not dried. In this embodiment, the crystalline lipopeptide is preferably stored in a solution that preserves the crystalline nature of the lipopeptide. Vials may be filled with the crystalline lipopeptide and solution under sterile or nonsterile conditions. In a preferred embodiment, the conditions are sterile. Alternatively, the crystalline lipopeptide and solution may be used to fill bulk packaging.

In a preferred embodiment the crystals are produced, and optionally dried, under sterile conditions for use as active ingredients in pharmaceutical compositions. Methods of sterile crystallization and sterile filtration as well as methods of sterilizing a final pharmaceutical product are known in the art. See, e.g., Remington: The Science and Practice of Pharmacy, Easton, Pa.: Mack Publishing Company (1995), pp. 1474-1487.

The pharmaceutical compositions of the invention comprise the crystalline form of daptomycin or A-21978C analog and a pharmaceutically-acceptable carrier, diluent or excipient as defined below. The pharmaceutical compositions of the invention are useful in treating diseases caused by gram-positive pathogens including, but not limited to, diseases such as complicated skin and soft tissue infections, community-acquired pneumonia, complicated urinary tract infections, enteroccocal infections, endocarditis and bacteremia. As used herein the phrase “therapeutically-effective amount” means an amount of daptomycin or A-21978C analog according to the present invention that prevents the onset, alleviates the symptoms, or stops the progression of a bacterial infection. The term “treating” is defined as administering, to a subject, a therapeutically-effective amount of a compound of the invention, both to prevent the occurrence of an infection and to control or eliminate an infection. The term “subject”, as described herein, is defined as a mammal, a plant or a cell culture. In a preferred embodiment, a subject is a human or other animal patient in need of lipopeptide treatment.

Formulations and Compositions

The crystalline daptomycin and A-21978C analogs (also referred to herein as “therapeutics” or “active compounds”) of the invention, and pharmaceutically acceptable derivatives or salts thereof, can be incorporated into pharmaceutical compositions suitable for administration. Such compositions typically comprise the active compound and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The active compounds disclosed herein can also be formulated as liposomes. Liposomes are prepared by methods known in the art, such as described in Epstein et al., Proc. Natl. Acad. Sci. USA, 82:3688 (1985); Hwang et al., Proc. Natl Acad. Sci. USA, 77:4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

A pharmaceutical composition of the invention is formulated to be compatible with its intended route of administration. Examples of routes of administration include parenteral (e.g., intravenous), intradermal, subcutaneous, oral, respiratory, (e.g., inhalation), transdermal (i.e., topical), transmucosal, and vaginal or rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); physiologically acceptable buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., daptomycin or related analog) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery and in the form of suppositories, gels or sponges for vaginal delivery. For example, the compounds can be incorporated into devices for administration vaginally, such as incorporation into a sponge as described in U.S. Pat. No. 5,527,534, or a dissolvable vehicle as described in U.S. Pat. No. 5,529,782.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In some embodiments, oral or parenteral compositions are formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Sustained-release preparations can be prepared, if desired. Suitable examples of sustained-release preparations include semipermeable matrices of solid hydrophobic polymers containing the active compound, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The pharmaceutical compositions can be included in a container, pack, or dispenser together with instructions for administration.

Those of ordinary skill in the art are well versed in determining the amounts of therapeutic compound to be administered in the practice of the methods of the invention. Such factors to consider about the subject to be treated, including sex, weight, type and extent of disorder, are typically analyzed in order to determine the correct dosage for treating the disorder. In a preferred embodiment for treating humans suffering from a disorder as defined above, the dosages are typically in the range of about 4 mg/kg to about 50 mg/kg.

Based on the disclosure of combining the crystalline lipopeptides into pharmaceutical compositions for ingestion or application to the body of a subject, those of ordinary skill will recognize that similar formulations can be prepared for incorporating the crystalline lipopeptides into various personal care compositions to make products including, without limitation, a washing formulation, soap, shampoo, deodorant, perfume, cologne, and antiperspirants.

The crystalline lipopeptides of the invention may also be administered in the diet or feed of a patient or animal. If administered as part of a total dietary intake, the amount of crystalline lipopeptide can be less than 1% by weight of the diet and preferably no more than 0.5% by weight. The diet for animals can be normal foodstuffs to which crystalline lipopeptide can be added or it can be added to a premix.

### EXAMPLES

The daptomycin used in these examples was obtained from Cubist Pharmaceuticals, Inc. (Cambridge, MA). The samples were obtained as a pale yellow amorphous powder, with a solubility at 25° C. of greater than 1 g/mL in water and a solubility of 2.8 mg/mL in ethanol. The amorphous daptomycin preparation was hygroscopic and decomposed at 215° C.

### Example 1

In a microbatch crystallization, 25 μL of a daptomycin stock (20 mg/mL in methanol) was sequentially mixed with 15 μL of reagent stock (200 mM calcium acetate, 0.1 M cacodylate (pH 6.5), 18% [w/v] PEG 8000 and 15 μL ethylene glycol) to give a solution that was 27.5% aqueous component, 45% methanol and 27.5% ethylene glycol. Urchin-like crystals were formed at a yield of 50% with a purity of 98%.

### Example 2

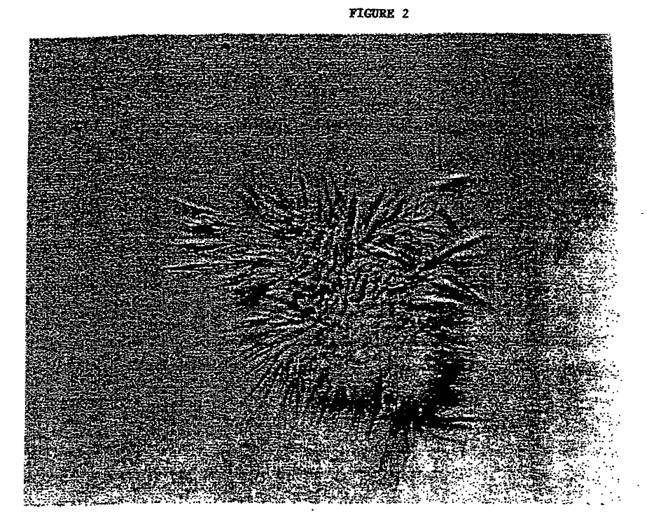

A daptomycin stock was prepared by dissolving 440 mg daptomycin in 1 mL of a buffer containing 25 mM sodium acetate (pH 5.0) and 5 mM CaCl₂. Crystallization was done by the vapor diffusion (hanging drop) method, in which 5 μL of the daptomycin stock was added to 5 μL of 0.1 M tri-sodium citratedihydrate (pH 5.6), and 35% [v/v] tert-butanol in water to form a drop. The drop was suspended over a reservoir solution (0.1 M tri-sodium citrate dihydrate (pH 5.6), and 35% [v/v] tert-butanol in water) in an air-tight environment until crystallization occurred. This method yielded urchin-like daptomycin crystals. See, e.g., FIG. 2.

### Example 3

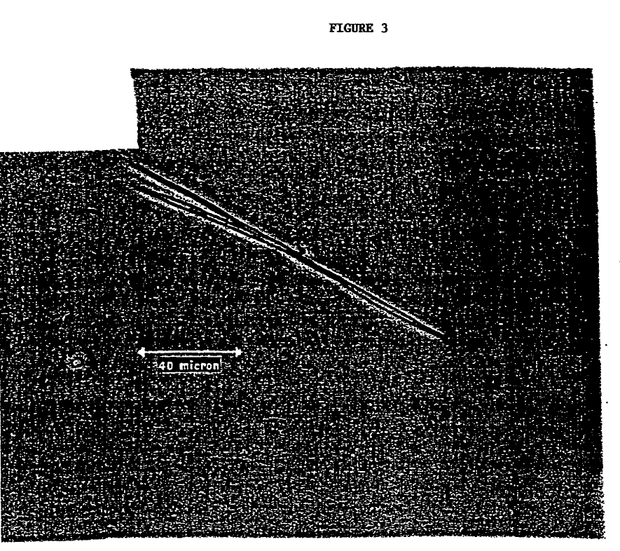

5 μL of a daptomycin stock prepared as in Example 2 was added to 5 μL of a solution containing 0.1 M sodium cacodylate (pH 6.5), 0.2 M calcium acetate and 9% [w/v] PEG 8000. Crystallization was done by the vapor diffusion method as described in Example 3. This method yielded needle-like daptomycin crystals. See, e.g., FIG. 3.

### Example 4

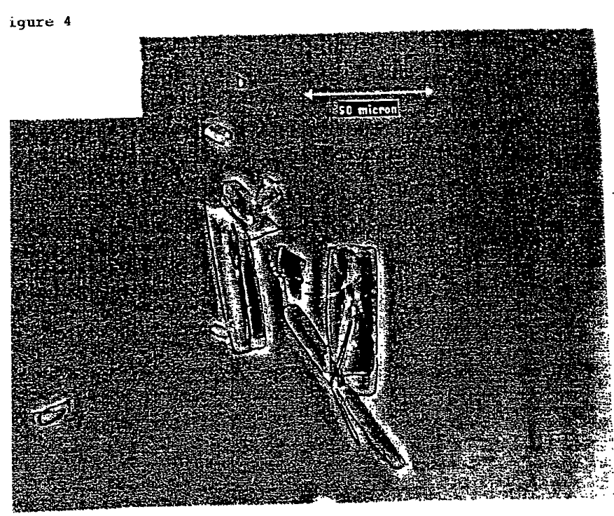

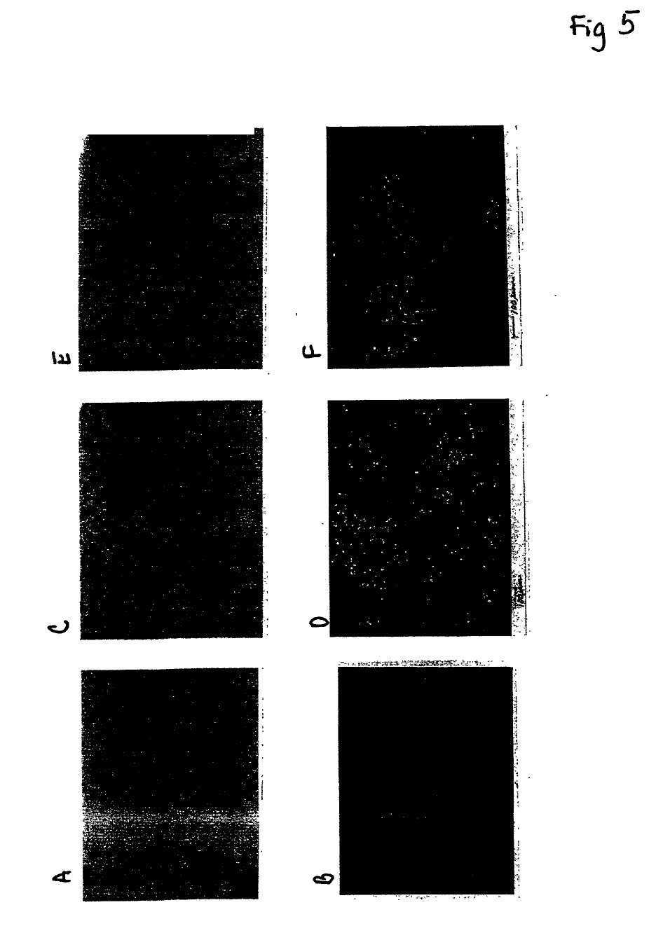

5 μL of a daptomycin stock prepared as in Example 2 was added to 5 μL of a solution of 0.1 M sodium cacodylate (pH 6.5), 0.2 M zinc acetate and 9% [w/v] PEG 8000 containing 0.1 μL benzamidine to give a final concentration of 220 mg/mL daptomycin. Crystallization was done by the vapor diffusion method as described in Example 3. This method yielded rod-like daptomycin crystals. See, e.g., FIG. 4.

### Example 5

One mL of daptomycin (97.1% pure as determined by HPLC) at a concentration of 20-25 mg/mL in water was sequentially mixed with 231 μL water, 77 μL of 1M calcium acetate (pH 6.0), 960 μL propylene glycol and 231 μL of 50% [w/v] PEG 4000. The solution was allowed to sit for 4-5 hours at 4° C. Urchin-like crystals were formed at a yield of 75%. The crystalline daptomycin was washed with isopropanol. The daptomycin was 98.4% pure as determined by HPLC.

### Example 6

Daptomycin (200 mg, 97.1% pure) was dissolved in 2.54 mL water. The daptomycin solution was sequentially mixed in order with 10.0 mL methanol, 0.78 mL 1 M calcium acetate (pH 6.0), 9.50 mL propylene glycol and 2.20 mL 50% [w/v] PEG 4000 to give a final volume of 25.02 mL. The mixture was tumbled at room temperature for 10-14 hours in a hematology mixer (Fischer). Crystals began to appear within a few hours. Final yield was approximately 70-80% after 14 hours. The crystals were harvested by centrifugation at 1000 rpm for 15 minutes. The supernatant was removed and the crystals were resuspended in 12.5 mL isopropanol. The daptomycin suspension was transferred to a borosilicate glass column (10×2.5 cm, 20 micron porosity) (Biorad) and the isopropanol was removed by allowing it to drip by gravity. The crystals were dried by a nitrogen stream. Any lumps were broken up during the drying procedure to obtain a uniform dry sample. Crystals prepared by this method were urchin-like and had a purity of 98.37%.

### Example 7

Daptomycin was crystallized according to Example 6 except that PEG 8000 was used in replacement of PEG 4000. The quantities of reagents used are identical to those in Example 6. Crystals prepared by this method were urchin-like and had a purity of 98.84%.

### Example 8

Daptomycin (400 mg) was dissolved in water. Sodium acetate was added to achieve a final concentration of 187 mM. Calcium chloride was added to achieve a final concentration of 28 mM. The daptomycin solution was mixed and isopropanol was added to a final concentration of 78.4%. The solution was mixed and incubated. A precipitated material was formed after incubation. The precipitated material appeared to be urchin-like crystals of approximately 60 im diameter by optical microscopy. The material was then dried. The dry material contained approximately 30-40% salt. After drying, powder x-ray diffraction was performed. The powder x-ray diffraction did not show the presence of crystals in the dried daptomycin precipitate.

### Example 9

One gram of daptomycin (approximately 91.5% purity) was added to 16.8 mL of distilled water and dissolved. 2.5 mL of 1M calcium acetate (pH 6.1) and 60 mL of isopropanol was added. The solution was placed in a 27° C. water bath and permitted to equilibrate to the temperature of the water bath. 5 mL aliquots of isopropanol were slowly added until the solution became cloudy (a total of approximately 30 mL isopropanol). The solution was incubated overnight at 27° C. to form a precipitate. The precipitate appeared to contain urchin-like crystals of approximately 60 m by optical microscopy. See FIG. 2.

The daptomycin precipitate was poured into a pressure filter/drying funnel and filtered by gravity. The precipitate was washed at room temperature twice with 25 mL each time of a washing solution (80% isopropanol and 20% solution A where solution A consists of 18 mL of water and 2 mL of glacial acetic acid) and allowed to drip by gravity overnight. The precipitate was then transferred to a desiccator and dried under vacuum. After drying, powder x-ray diffraction was performed. The powder x-ray diffraction did not show the presence of crystals in the dried daptomycin precipitate. However, purity analysis of the precipitated material by HPLC showed that the material was 98.2% pure daptomycin. Significantly, the daptomycin preparation after precipitation has significantly less of the anhydrous daptomycin than the daptomycin preparation before precipitation.

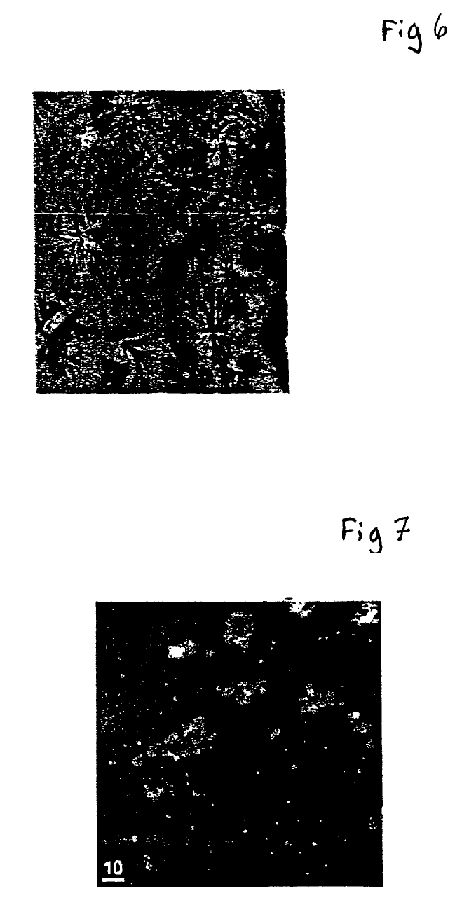

Without wishing to be bound by any theory, applicants believe that the conditions used to precipitate the daptomycin in Examples 8 and 9 actually produce a crystalline form of daptomycin but that the subsequent washing steps and/or drying steps cause the crystalline daptomycin to revert to a non-crystalline form. Nonetheless, the formation of crystalline daptomycin in solution is shown in the photomicrograph of FIG. 6 and by the birefringence of a crystal sample in polarized light (FIG. 7).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. Daptomycin in crystalline form.

2. A lipopeptide in crystalline form wherein the lipopeptide is chosen from the group consisting of daptomycin and A-21978C analogs.

3. The crystalline lipopeptide of claim 2, wherein the crystals are needle-like, rod-like, needle cluster or urchin-like, flake-like, or plate-like.

4. The crystalline lipopeptide of claim 3, wherein the crystals are urchin-like or needle-like.

5. The crystalline lipopeptide of claim 2 having a purity of at least 95%.

6. The crystalline lipopeptide of claim 2 having a purity of at least 97%.

7. A pharmaceutical composition comprising the crystalline lipopeptide of claim 2 and a pharmaceutically acceptable carrier.

8. The pharmaceutical composition of claim 7, wherein the crystalline lipopeptide is daptomycin.

9. The pharmaceutical composition of claim 7, wherein the crystalline lipopeptide is enterically coated for oral administration.

10. The pharmaceutical composition of claim 7, which is in the form of micronized particles or microspheres.

11. A container comprising the pharmaceutical composition of claim 7.

12. A formulation comprising the crystalline lipopeptide of claim 2.

13. A container comprising the formulation of claim 12 and a physiologically acceptable buffer.

14. A composition of matter comprising the formulation of claim 12, selected from the group consisting of a pharmaceutical composition, a food composition, a feed composition, a veterinary composition, a cosmetic composition or a personal care composition.

15. The composition of matter of claim 14, wherein the composition is a personal care composition which is chosen from the group consisting of washing formulation, soap, shampoo, deodorant, perfume, cologne, or antiperspirant.

16. A method of preparing a crystalline form of a lipopeptide which comprises combining the lipopeptide with a crystallization solution comprising at least one cation and at least one alcohol chosen from the group consisting of polyhydric alcohols and low molecular weight alcohols and combinations thereof, wherein the lipopeptide is chosen from the group consisting of daptomycin and daptomycin analogs.

17. The method of claim 16, wherein the polyhydric alcohol is chosen from the group consisting of ethylene glycol, propylene glycol, glycerol, 1,2-propane diol, 2-methyl-2,4-pentanediol, 1,6-hexanediol, and 1,4-butanediol.

18. The method of claim 16, wherein the low molecular weight alcohol is chosen from the group consisting of methanol, isopropanol, tert-butanol, and n-propanol.

19. The method of claim 16, wherein the cation is a divalent cation.

20. The method of claim 19, wherein the divalent cation is chosen from the group consisting of manganese, magnesium, and calcium.

21. The method of claim 20, wherein the divalent cation is calcium.

22. The method of claim 16, wherein the crystallization solution consists of at least one salt and at least one low molecular weight alcohol.

23. The method of claim 16, wherein the crystallization solution further comprises one or more additional components chosen from the group consisting of organic precipitants, pH buffers, low molecular weight alcohols and detergents.

24. The method of claim 23 wherein the crystallization solution comprises as an organic precipitant polyethylene glycol.

25. A method for treating a disease caused by a gram-positive pathogen in a subject which comprises administering to the subject the pharmaceutical composition of claim 7 which comprises the crystalline lipopeptide in a therapeutically effective amount.

26. The method of claim 25 wherein the disease is chosen from the group consisting of complicated skin and soft tissue infections, community-acquired pneumonia, complicated urinary tract infections, enteroccocal infections, endocarditis and bacteremia.

27. A method for administering to a subject in need thereof a crystalline lipopeptide or salt thereof, wherein the crystalline lipopeptide is chosen from the group consisting of crystalline daptomycin and crystalline A-21978C analogs, which comprises administering to the subject a pharmaceutical composition comprising the crystalline lipopeptide or salt thereof and a pharmaceutically acceptable carrier.

28. The method of claim 27 wherein the crystalline lipopeptide or salt thereof is administered to the subject by pulmonary administration as a micronized particle.

29. The method of claim 27 wherein the crystalline lipopeptide or salt thereof is administered to the subject as a sustained release form.

30. The method of claim 27 wherein the crystalline lipopeptide is administered orally.

31. The method of claim 27 wherein the crystalline lipopeptide is administered subcutaneously.

32. The method of claim 27 wherein the crystalline lipopeptide is administered intravenously.

33. The method of claim 27 wherein the crystalline lipopeptide is administered intramuscularly.

34. A method of storing a lipopeptide selected from the group consisting of daptomyin and A-21978C analogs which comprises preparing the lipopeptide in crystalline form and storing the crystalline lipopeptide.

35. In a method for preparing a lipopeptide selected from the group consisting of daptomycin and A-21978C analogs the improvement which comprises preparing the lipopeptide in crystalline form.
